(19) United States
(12) Patent Application Publication
(10) Pub. No.: US 2003/0170702 A1
(43) Pub. Date: Sep. 11, 2003

(54) Methods for polysome distribution analysis of mRNA's

(76) Inventor: Bryan Hoffman, Kimberton, PA (US)

Correspondence Address: CLARK & ELBING LLP, 101 FEDERAL STREET, BOSTON, MA, 02110, US

(21) Appl. No.: 10/342,749

(22) Filed: Jan. 15, 2003

Related U.S. Application Data
(60) Provisional application No. 60/349,116, filed on Jan. 16, 2002.

(51) Int. Cl. C12Q 1/68; C07H 21/02; C12P 19/34

(52) U.S. Cl. 435/6; 435/91.2; 536/25.4

(57) ABSTRACT

The invention provides methods for measuring the translation efficiency of an RNA of interest and methods for identifying a candidate compound that modulates the translation efficiency of the RNA.

### CROSS-REFERENCE TO RELATED APPLICATION

This application claims the benefit of the filing date of U.S. provisional application 60/349,116, filed Jan. 16, 2002.

### BACKGROUND OF THE INVENTION

In general, the invention features improved methods for measuring mRNA translation efficiency and for identifying compounds that modulate the translation efficiency of an mRNA.

One mechanism by which protein expression is regulated is through modulating translation efficiency. In eukaryotes, translational regulation usually occurs at the initiation step. Therefore, cis-elements in the 5′ untranslated region (UTR) are important for overall regulation of protein synthesis. There is also a growing body of evidence that supports the role of the poly(A) tail and elements in the 3′ UTR in regulating initiation of translation. Compounds that inhibit the translation of an mRNA associated with a disease state are useful in the prevention and treatment of the disease. Similarly, compounds that promote the translation of an mRNA associated with a beneficial effect, such as the prevention of a disease state, are therapeutically useful.

Improved methods are needed to more rapidly and more accurately measure the translation efficiency of an mRNA and to select compounds that modulate this translation efficiency.

### SUMMARY OF THE INVENTION

The present invention provides improved methods for measuring the translation efficiency of an mRNA of interest by determining the polysome distribution of the mRNA. In particular, the methods involve purifying an RNA molecule of interest from a polysome distribution gradient by removing most or all of the heparin and/or sucrose from the polysome distribution gradient fractions. The reduced level of heparin and/or sucrose increases the efficiency of the subsequent amplification (e.g., RT-PCR amplification) of the RNA molecule of interest in each fraction, enabling the amount of RNA to be rapidly and accurately determined in a single-step RT-PCR reaction. The amount of RNA contained in the same sucrose gradient fraction as high molecular weight (HMW) polysomes is directly proportional to the level of translation efficiency of the RNA (i.e., the greater the amount of RNA associated with HMW polysomes, the greater the translation efficiency). These methods can also be used to determine the effect of a candidate compound on the translation efficiency of the RNA by incubating the RNA in the presence of the candidate compound and measuring the change in the polysome distribution of the RNA that is mediated by the candidate compound.

Accordingly, in one such aspect, the invention provides a method of amplifying an RNA molecule from a polysome distribution sucrose gradient. This method involves purifying an RNA molecule in a solution from a polysome distribution sucrose gradient under conditions that reduce the amount of heparin in the solution by at least 60, 70, 80, 90, 95, 98, or 100% and amplifying the purified RNA molecule. In various embodiments, the method also includes quantitating the amount of the amplified RNA molecule and/or sequencing the amplified RNA molecule. Desirably, the amount of sucrose in the solution is decreased by at least 60, 70, 80, 90, 95, 98, or 100% prior to amplifying the purified RNA molecule. In desirable embodiments, the amount of heparin remaining after the purification step is less than 0.018, 0.015, 0.012, 0.008, 0.004 ng/μL and/or the amount of sucrose remaining after the purification step is less than 0.006, 0.004, 0.002, or 0.001% (w/v) sucrose. In various embodiments, the amount of heparin and the amount of sucrose in the solution are decreased simultaneously. In other embodiments, the purification step does not involve an extraction or precipitation step. Desirably, the amount of the RNA in one or more fractions from the sucrose gradient indicates the translation efficiency of the RNA. In some embodiments, the polysome distribution analysis is performed in the presence of 1, 3, 5, 10, 20, 50, 100 or more candidate compounds. A change in the polysome distribution of the RNA effected by a candidate compound indicates that the candidate compound modulates the translation efficiency of the RNA. For example, an increase in the amount of RNA associated with HMW polysomes indicates that the candidate compound increases the translation efficiency of the RNA, and a decrease in the amount of RNA associated with HMW polysomes indicates that the candidate compound decreases the translation efficiency. In various embodiments, the candidate compound directly or indirectly binds the RNA molecule. In other embodiments, at least 2, 5, 10, 30, 50, 75. 100, or more RNA molecules are amplified. In some embodiments, the RNA molecule is a eukaryotic RNA molecule, such as a mammalian or human RNA molecule. Desirably, the RNA molecule applied to the sucrose gradient is isolated from a cell (e.g., a eukaryotic, mammalian, or human cell) that is stably or transiently transfected with a nucleic acid (e.g., cDNA or DNA molecule) corresponding to the RNA molecule. In other embodiments, the RNA molecule contains a portion or all of a 5′ and/or 3′ untranslated region (UTR) of an RNA of interest operably linked to a reporter gene.

The methods of the invention for removing heparin and/or sucrose from a nucleic acid-containing solution can be used to remove heparin and/or sucrose from a solution prior to amplifying a nucleic acid bound to a compound of interest. These methods facilitate the identification of nucleic acids that bind a compound of interest and the identification of candidate compounds that modulate the binding of the nucleic acids to the compound of interest.

In one such aspect, the invention provides a method of amplifying a nucleic acid that binds a compound of interest. This method involves contacting one or more nucleic acids (e.g., mRNA, tRNA, snRNA, DNA, or cDNA) in a solution including heparin with a compound of interest under conditions that allow at least one nucleic acid to bind the compound of interest, and purifying the nucleic acid that binds the compound of interest under conditions that reduce the amount of heparin in the solution by at least 60, 70, 80, 90, 95, 98, or 100%. The purified nucleic acid is amplified. In some embodiments, the method also includes quantitating the amount of the amplified nucleic acid and/or sequencing the amplified nucleic acid. In desirable embodiments, the amount of heparin remaining after the purification step is less than 0.018, 0.015, 0.012, 0.008, 0.004 ng/μL and/or the amount of sucrose remaining after the purification step is less than 0.006, 0.004, 0.002, or 0.001% (w/v) sucrose. In other embodiments, the purification step does not involve an extraction or precipitation step. In various embodiments, the compound of interest is an organic molecule having a molecular weight less than 2000, 1000, 750, or 500 daltons. In other embodiments, the compound of interest is a protein, such as an RNA or DNA binding protein. In some embodiments, the contacting of the nucleic acid with the compound of interest is performed in the presence of 1, 3, 5, 10, 20, 50, 100 or more candidate compounds. A change in the amount of the nucleic acid bound to the compound of interest that is effected by a candidate compound indicates that the candidate compound modulates the binding of the nucleic acid to the compound of interest. In various embodiments, the compound of interest or candidate compound directly or indirectly binds the nucleic acid. In other embodiments, at least 2, 5, 10, 30, 50, 75, 100, or more nucleic acids are amplified. In some embodiments, at least 2, 5, 10, 30, 50, 75, 100, or more compounds of interest and/or candidate compounds are contacted with a nucleic acid. In some embodiments, the nucleic acid is a eukaryotic RNA or DNA molecule, such as a mammalian or human RNA or DNA molecule. In other embodiments, the RNA molecule contains a portion or all of a 5′ and/or 3′ untranslated region (UTR) of an RNA of interest operably linked to a reporter gene.

It is also contemplated that the methods of the invention for removing heparin and/or sucrose from a solution can be used to purify nucleic acids, such as mRNA, tRNA, snRNA, DNA, or cDNA molecules or a combination thereof, from any other solution containing heparin and/or sucrose.

By “translation” is meant the process of generating a protein that has an amino acid sequence dictated by the codon sequence of an mRNA that encodes the protein.

By “translation efficiency” is meant the ability of an RNA to be translated into a protein. Translation efficiency can be measured, for example, by polysome distribution analysis as described herein. A Polysome is the assemblage of an mRNA, one or more ribosomes, and the protein being translated. The greater the number of ribosomes in the polysomes and thus the greater the molecular weight of the polysomes, the more efficiently the mRNA is translated. Thus, an RNA with a high level of translation efficiency is associated with high molecular weight polysomes. Alternatively, an RNA is determined to have a low level of translation efficiency if (i) the RNA is not associated with high molecular weight polysomes, (ii) a smaller amount of the RNA is associated with high molecular weight polysomes, or (iii) the RNA is associated with low molecular weight polysomes. If desired, translation efficiency can be confirmed by determining the level of protein expression or activity. This confirmation may be performed in combination with the detection of steady state RNA levels to confirm that a modulation in protein expression or activity is a result of a change in RNA translation efficiency.

Desirably, the level of RNA translation efficiency in a cell or in vitro sample contacted with a compound that decreases the level of translation efficiency is decreased by at least 10%, 30%, 40%, 50%, 75%, or 90% relative to a control cell or sample that is not contacted with the compound, that is contacted with the compound vehicle only, or that is contacted with a control compound known to have negligible effect on the translation efficiency. Desirably, the level of RNA translation efficiency in a cell or in vitro sample contacted with a compound that increases the level of translation efficiency is increased by at least 1.5-fold to 2-fold, more desirably by at least 3-fold to 5-fold, and most desirably by at least 10-fold to 20-fold, relative to a control cell or sample that is not administered the compound, that is contacted with the compound vehicle only, or that is contacted with a control compound known to have negligible effect on the translation efficiency.

By “modulates” is meant changes the level of translation efficiency, either by decreasing or increasing the translation efficiency.

By “reporter gene” or “reporter nucleic acid” is meant any gene or translatable nucleotide sequence that encodes a product whose RNA or protein expression is detectable and/or quantitatable by immunological, chemical, biochemical, or biological assays. A reporter gene may be detected at the nucleic acid level by detecting nucleic acid expression, for example, by Northern blot analysis, or filter binding assays. A reporter gene product may, for example, have one of the following attributes, without restriction: fluorescence (e.g., green fluorescent protein), enzymatic activity (e.g., β-galactosidase, luciferase, or chloramphenicol acetyltransferase), toxicity (e.g., ricin), or an ability to be specifically bound by a second molecule (e.g., biotin or a detectably labeled antibody). It is understood that any engineered variants of reporter genes that are readily available to one skilled in the art, are also included, without restriction, in the foregoing definition. In addition, a reporter gene is any nucleic acid sequence that is not endogenously contained as an RNA sequence of the cell or sample of interest.

By a “UTR” is meant a nucleic acid derived from the 5′ or 3′ untranslated region of a gene that desirably contains 10, 25, 50, 75, 100, 150, or more of the contiguous nucleotides that are outside of the coding region of a gene of interest. Desirably, the UTR is the full length UTR sequence of a gene of interest, for example, the TNFα gene.

By “operably linked” is meant that a gene and one or more regulatory sequences are connected in such a way as to permit gene expression when the appropriate molecules (e.g., transcriptional activator proteins) are bound to the regulatory sequences. As used herein, an RNA comprising a regulatory element is operably linked to a promoter and/or 5′ UTR sequences and/or 3′ UTR sequences that direct transcription and/or translation of a reporter gene.

By “promoter” is meant a minimal sequence sufficient to direct transcription.

By “regulatory element” is meant sequences that can modulate expression of a gene or gene product. Examples of regulatory sequences include, but are not limited to promoters, enhancers, sequences that stabilize an RNA sequence, sequences that enhance protein stability, translation termination sequences, and additional 5′ or 3′ UTR sequences.

By “expression vector” is meant a DNA construct that contains regulatory elements, for example, the UTR sequences of the present invention and a promoter that are operably linked to a downstream gene. Transfection of the expression vector into a recipient cell allows the cell to express RNA encoded by the expression vector. An expression vector may be a genetically engineered plasmid or virus, derived from, for example, a bacteriophage, adenovirus, retrovirus, poxvirus, herpes virus, or artificial chromosome.

By “candidate compound” or “compound of interest” is meant a chemical, be it naturally-occurring or artificially-derived, that is surveyed for its ability to modulate RNA translation efficiency, by employing one of the assay methods described herein. Test compounds may include, for example, peptides, proteins, synthetic organic molecules, naturally occurring organic molecules, nucleic acid molecules, and components thereof.

By a “derivative” is meant a structural derivative having a chemical modification of a compound that enhances the bioavailability, solubility, or stability in vivo or ex vivo, or that reduces the toxicity or dosage required. Desirably, the derivative has at least 70, 80, 90, or 100% of the ability to modulate the translation efficiency of an RNA as the parent compound from which it was derived. Such modifications are known to those skilled in the field of medicinal chemistry.

By “transformation” or “transfection” is meant any method for introducing foreign molecules into a cell (e.g., a bacterial, yeast, fungal, algal, plant, insect, or animal cell, particularly a mammalian cell). Lipofection, DEAE-dextran-mediated transfection, microinjection, protoplast fusion, calcium phosphate precipitation, retroviral delivery, electroporation, and biolistic transformation are just a few of the methods known to those skilled in the art which may be used. In addition, a foreign molecule can be introduced into a cell using a cell penetrating peptide, as described, for example, by Fawell et al. (Proc. Natl. Acad. Sci. USA 91:664-668 (1994)) and Lindgren et al. (TIPS 21:99-103 (2000)).

By “transformed cell” or “transfected cell” is meant a cell or a descendent of a cell into which a nucleic acid molecule has been introduced, by means of recombinant nucleic acid techniques. Such cells may be either stably or transiently transfected.

By “protein” or “polypeptide” is meant any chain of more than two amino acids, regardless of post-translational modification (e.g., glycosylation or phosphorylation), constituting all or part of a naturally-occurring protein or peptide, or constituting a non-naturally occurring protein or peptide.

The present invention provides a number of advantages related to measuring the translation efficiency of an mRNA of interest. These methods are more accurate than current methods that require multiple amplification steps to produce enough amplified RNA for quantitation. These methods also take significantly less time to perform than current RNA precipitation or extraction methods for purifying RNA. The high through put methods of the invention facilitate the screening of libraries of candidate compounds to identify compounds that modulate the translation efficiency of an mRNA of interest. The selected modulates are useful in the development of therapeutics for the prevention or treatment of a disease or condition associated with the mRNA.

Other features and advantages of the invention will be apparent from the following detailed description.

### BRIEF DESCRIPTION OF THE DRAWINGS

FIG. 1A is a graph of the induction of TNFα RNA in response to LPS that was measured in three days using the methods of the present invention.

FIG. 1B is a graph of the induction of TNFα RNA in response to LPS that was measured in three weeks using traditional two-step precipitation and Northern blot analysis of a polysome distribution gradient.

FIG. 2 is a graph showing the amount of actin RNA following LPS treatment that was measured in three days using the methods of the present invention. This analysis was performed on the same samples analyzed in FIG. 1A.

### DETAILED DESCRIPTION

We have developed novel methods for purifying nucleic acids, such as RNA and DNA, prior to reverse transcription, polymerase chain reaction, or other nucleic acid amplification methods. These methods reduce the level of sucrose (e.g., sucrose from a polysome distribution sucrose gradient) and/or heparin (e.g., heparin added to inhibit cleavage of RNA by RNase or to inhibit non-specific binding to RNA) in the solution containing the nucleic acids. As we discovered, sucrose and heparin inhibit nucleic acid amplification methods such as RT-PCR. Thus, removing these components prior to RT-PCR increases the efficiency of RT-PCR and enables a low concentration of RNA to be amplified and quantitated.

For example, methods have been developed to purify an RNA molecule of interest from a polysome distribution gradient by removing most or all of the heparin and/or sucrose from the polysome distribution gradient fractions. Heparin and/or sucrose can be removed, for example, by applying the solution to a Qiagen column and treating the Qiagen column using the modified method described herein. The reduced level of heparin and/or sucrose increases the efficiency of the subsequent RT-PCR amplification of the RNA molecule of interest in each fraction, enabling the amount of RNA to be rapidly and accurately determined in a single-step RT-PCR reaction. The amount of RNA contained in the same sucrose gradient fraction(s) as high molecular weight (HMW) polysomes is directly proportional to the level of translation efficiency of the RNA (i.e., the greater the amount of RNA associated with HMW polysomes the greater the translation efficiency). By adding a ³²P-labeled RNA to the individual fractions of a sucrose gradient, we demonstrated that the present method recovers an equal amount of the labeled RNA from the different fractions. This result indicates that the recovery of a specific RNA is consistent across a sucrose gradient in which the composition of each fraction differs greatly in not only the amount of sucrose but also the amount of cellular protein present.

In contrast, current two-step precipitation or phenol extraction methods do not significantly reduce the level of heparin, and thus often do not generate RNA that can be analyzed by RT-PCR. The Trizol (Life Technologies) RNA purification reagent also did not remove heparin from an RNA solution. Additionally, the Strataprep (Stratagene) and the SV RNA Isolation system (Promega) RNA purification systems did not remove a sufficient amount of sucrose from an RNA-containing solution to allow RT-PCR.

The present methods for removing heparin and/or sucrose from an RNA-containing solution can also be used to determine the effect of a candidate compound on the translation efficiency of the RNA by incubating the RNA in the presence of the candidate compound and measuring the change in the polysome distribution of the RNA that is mediated by the candidate compound. In addition, the present methods can be used to determine whether a UTR from a gene of interest modulates translation efficiency. For example, a UTR from an RNA of interest can be operably linked to a reporter gene and the effect of the UTR on the polysome distribution of the resulting RNA can be determined.

Improved methods are also provided for identifying an RNA molecule that binds to a compound of interest. In these methods, one or more RNA molecules are contacted with one or more compounds of interest, and complexes containing an RNA bound to a compound of interest are isolated using standard methods, such as gel filtration or affinity chromatography. The RNA is then purified under conditions that reduce the amount of heparin, which is often added to inhibit RNase cleavage or inhibit non-specific binding, in the solution. The purified RNA can then be amplified using standard methods. If desired, candidate compounds can be added to the solution containing the RNA and the compound of interest to determine whether the candidate compounds promote or inhibit the interaction between the RNA and the candidate compounds.

These methods are described further below.

### EXAMPLE 1

Inhibition of Nucleic Acid Amplification by Heparin and Sucrose

We discovered that heparin and sucrose inhibit reverse transcription (RT) and polymerase chain reaction (PCR). To determine the level of heparin or sucrose that inhibits these reactions, RT and PCR reactions were performed at varying heparin and sucrose concentrations. Each reaction mixture contained 25 μL 2× SYBR Green PCR Master Mix (Applied Biosystems Inc), 0.25 μL MultiScribe Reverse Transcriptase at 50U/μl (ABI), 0.4 μL RNase Inhibitor at 20U/μl (ABI), 25 nM forward primer, 25 nM reverse primer, 10 μl RNA template, and enough water to bring the final reaction volume to 50 μl. For the RT reactions, E coli RNase P RNA was used as the RNA template, and 1.25 μl of each primer from a stock solution of 1 μM was used, giving a final primer concentration of 25 nM. The RNase P primers were RNASEP-F19 (5′-GCCGCTTCGTCGTCGTCCTCTTC-3′, SEQ ID NO: 1) and RNASEP-R278 (5′-GGCCGTACCTTATGAACCCCTATTTGG-3′, SEQ ID NO: 2). All primers were purchased from Integrated DNA Technologies, Inc. The 2× SYBR Green Master Mix contains Taq polymerase, nucleotides, buffer, and magnesium. These reaction mixtures were assembled in a Micro Amp Optical 96 well reaction plate (ABI).

To examine the reverse transcription efficiency in the presence of sucrose and heparin, the reverse primer specific for RNase P RNA was ³²P labeled, and used as the only source of reverse primer. The radiolabeled primer was extended using reverse transcription during an incubation at 48° C. for 30 minutes. The resultant DNA was isolated and separated on a 6% denaturing gel. After soaking and drying the gel, the ³²P labeled reverse transcription product was quantitated using the Cyclone Storage Phosphor System (Packard) and Optiquant Image Analysis Software (Packard). This software reports the intensity of a selected band on the gel in units called Digital Light Units (DLU). The number of DLUs is listed in the “Raw Activity” column in Table 1. The amounts of sucrose and heparin in the samples as well as the final concentration of sucrose (percent weight per volume, % w/v) and heparin in the RT reaction are also listed. The highest amount represents 30% sucrose (average of the amount in a typical sucrose gradient) and 1 mg/mL heparin (amount of heparin in a typical sucrose gradient fraction). The amount of reverse transcription activity (“% RT” column) is expressed as a percent of the activity in the presence of differing amounts of sucrose or heparin relative to the activity in the absence of these substances. The results indicate that sucrose does not inhibit the efficiency of reverse transcription under these conditions and that 0.32 ng/μL or more heparin significantly inhibits reverse transcription efficiency.

TABLE 1
Inhibition of RT by Heparin and Sucrose

| % Sucrose | Sucrose | Raw | | Heparin | Heparin | Raw | |
|---|---|---|---|---|---|---|---|
| of samples | (% W/V)_{f} | Activity | % RT | in samples | (ng/μL)_{f} | Activity | % RT |
| 30 | 1.2 | 5.72 | 96.1345 | 1000 | 40 | 0.08 | 0.8008 |
| 10 | 0.4 | 7.1 | 119.328 | 200 | 8 | 0.06 | 0.6006 |
| 3.33 | 0.133 | 9.27 | 155.798 | 40 | 1.6 | 0.36 | 3.6036 |
| 1.11 | 0.044 | 9.51 | 159.832 | 8 | 0.32 | 7.25 | 72.573 |
| 0.37 | 0.014 | 10.23 | 171.933 | 1.6 | 0.064 | 9.32 | 93.293 |
| 0.12 | 0.004 | 9.43 | 158.487 | 0.32 | 0.012 | 10.17 | 101.8 |
| 0.04 | 0.001 | 7.94 | 133.445 | 0.06 | 0.002 | 10.79 | 108.01 |
| 0 | 0 | 5.95 | 100 | 0 | 0 | 9.99 | 100 |

PCR reactions were also performed in the presence of varying amounts of heparin and sucrose. The plasmid pJA2′, which contains the DNA copy of the E coli RNase P RNA, was used as the template in these PCR reactions to generate both experimental and standard curves. Thirty-five cycles of PCR were performed in which the reaction mixture was incubated at 95° C. for 15 seconds for denaturation and 60° C. for one minute for annealing and extension. The reaction mixtures in the 96 well plate were loaded in the Gene Amp 5700 Sequence Detection System (ABI). This machine quantitates the amount of PCR product made after each cycle. A standard curve is generated with each reaction and used to calculate the quantity of the PCR product or amplicon for each experimental reaction. For the “Raw Activity” column in Table 2 the units are ng of DNA.

TABLE 2
Inhibition of PCR by Heparin and Sucrose

| % Sucrose | Sucrose | Raw | | Heparin | Heparin | Raw | |
|---|---|---|---|---|---|---|---|
| of samples | (% w/v)_{f} | Activity | % PCR | in samples | (ng/μL)_{f} | Activity | % PCR |
| 30 | 1.2 | 7.26 | 72.6 | 1000 | 40 | 0 | 0 |
| 10 | 0.4 | 6.67 | 66.7 | 200 | 8 | 0 | 0 |
| 3.33 | 0.133 | 6.57 | 65.7 | 40 | 1.6 | 0 | 0 |
| 1.11 | 0.044 | 6.1 | 61 | 8 | 0.32 | 0 | 0 |
| 0.37 | 0.014 | 6.7 | 67 | 1.6 | 0.064 | 3.1 | 29.808 |
| 0.12 | 0.004 | 6.66 | 66.6 | 0.32 | 0.012 | 7.9 | 75.962 |
| 0.04 | 0.001 | 10.2 | 102 | 0.06 | 0.002 11.2 | 107.69 | |
| 0 | 0 | 10 | 100 | 0 | 0 | 10.4 | 100 |

These results indicate that PCR is much more sensitive to sucrose and heparin than reverse transcription. Under these conditions, PCR efficiency is inhibited by 0.004 percent weight per volume (% w/v) sucrose and 0.012 ng/μL heparin.

### EXAMPLE 2

Quantitative Polysome Distribution Analysis of an RNA of Interest.

Polysome distribution analysis can be used to measure the translation efficiency of RNA isolated from stably transfected, transiently transfected, or untransfected cells or an in vitro sample. The amount of RNA associated with high molecular weight (HMW) polysomes (i.e., contained in the same sucrose gradient fraction) is directly proportional to the level of translation efficiency, with high amounts of RNA associated with the HMW polysomes indicating high translation efficiency of the RNA of interest. If an RNA mediates a lower level of translation efficiency, this effect is seen by distribution of the RNA throughout the sucrose gradient, or by the association of the RNA with only low molecular weight (LMW) polysomes, which are contained in a different fraction than the HMW polysomes.

To measure the translation efficiency of TNFα after LPS treatment, cellular RNA was isolated from THP-1 cells treated with LPS using standard methods and centrifuging through a sucrose gradient, as previously described (see, for example, U.S. Ser. No. 60/278,902, filed Mar. 26, 2001; U.S. Ser. No. 09/560,174, filed Apr. 28, 2000; Johannes and Sarnow, RNA 4:1500-1513, 1998).

For the purification of the mRNA in each fraction of the sucrose gradient, the RNeasy Mini Kit (Qiagen) was used. For this purification, steps from the “RNeasy Mini Protocol for RNA Cleanup” protocol were combined with steps from the “RNeasy Mini Protocol for the Isolation of Total RNA from Animal Cells,” as described below (RNeasy® Mini Handbook, Qiagen, May 1999). To each fraction from the sucrose gradient, 1 ng E. coli RNase P RNA was added as a control for RNA quantitation. This exogenous synthetic RNA is used to account for any small variability in RNA recovery. Next, 10 μl BME was added to each 1 ml of Buffer RLT needed. To a 100 uL aliquot of each sucrose gradient fraction, was added 350 μL of Buffer RLT. The resulting solution was mixed thoroughly. Then, 250 μL ethanol was added to the lysate and mixed well by pipetting. The sample (700 μL) was applied to a spin column sitting in a collection tube and spun for 15 seconds at over 10,000 rpm at room temperature. Alternatively, a 200 μL aliquot of each fraction can be used with twice the amount of buffer to keep the proper ratio of sample to buffer. In this case, half of the mixture is added to the column and centrifuged, and then the other half is added and centrifuged. Next, 700 μL of Buffer RW1 is pipetted onto the column and spun for 15 seconds at over 10,000 rpm to wash the column. The flow through and collection tube were discarded, and the column was transferred to a new collection tube. Buffer RPE (500 μl) was pipetted onto the column and spun for 15 seconds at over 10,000 rpm to wash. Next 500 μL of Buffer RPE was pipetted onto the column and spun for two minutes at maximum speed to dry the membrane. The column was placed into a new tube and spun one minute at full speed to ensure that no ethanol remained. The column was transferred to a new 1.5 mL collection tube, and 50 μl Rnase-free water at 37° C. was pipetted directly onto the membrane. The column was then spun for one minute at over 10,000 rpm to elute the RNA.

A similar high through-put method may be used to purify the RNA using a 96 column plate (RNeasy® 96 Handbook, Qiagen, February 1999). As described above, 1 ng RNase P or any other control RNA is added to each fraction for RNA quantitation. BME is added to the Buffer RLT at a ratio of 1 μL of BME per 10 mL Buffer RLT. A 100 μL aliquot of each fraction is mixed thoroughly with 350 μl of Buffer RLT. Ethanol (250 μL) is added to the lysate and mix well by pipetting. The sample (700 μL) is applied to a 96 column plate sitting in a vacuum manifold. A vacuum is applied for 15-60 seconds by sealing any unused wells with a plastic strip. Buffer RW1 (1 mL) is pipetted into each well of the 96-well plate. After five minutes, a vacuum is applied for 30 seconds. RPE (1 mL) is added into each well of the 96-well plate, and a vacuum is applied for 30 seconds. Another 1 mL of RPE is added to each well, and a vacuum is applied for 30 seconds. The plate tips are tapped onto a stack of paper towels (at least ¼″ thick) until no further liquid is released onto the towels. The vacuum apparatus is reassembled, and a vacuum is applied for 10 minutes. Water (80 μL) is added to each well, and incubated for one minute. The vacuum apparatus is reassembled to collect samples in a 96 well plate, and a vacuum is applied until transfer is complete. The elution step is repeated with another 80 μl of H₂O to elute any remaining RNA.

The amount of TNFα, actin, and control RNase P RNA purified from each sucrose gradient fraction was measured essentially as described in Example 1 with the RT and PCR reactions occurring in the same reaction mixture. The following primers were used for RT-PCR analysis of TNFα: TNFhF1848 (5′-AAGCCTGTAGCCCATGTTGTAGCA-3′, SEQ ID NO: 3) and TNFhR2470 (5′-TGATGGCAGAGAGGAGGTTGACCTTG-3′, SEQ ID NO: 4). For the RT-PCR analysis of actin, primers Actin-for (5′-TCACCCACACTGTGCCCATCTACGA-3′, SEQ ID NO: 5) and Actin-rev (5′-CAGCGGAACCGCTCATTGCCAATGG-3′, SEQ ID NO: 6) were used. To examine TNF, 2.5 μl of each the forward and reverse primers from a 1 μM stock solution was used to yield a final concentration of 50 nM in the 50 μl RT-PCR reaction. For actin, 5 μl of 1 μM stock solutions were used to generate a final concentration of 100 nM in the 50 μl RT-PCR reaction. RT-PCR was also performed to measure the amount of the control RNase P RNA, allowing the amount of the cellular TNFα and actin mRNA relative to the control RNase P mRNA to be calculated for each fraction. The thermal cycling parameters of the initial RT reaction and subsequent PCR reactions are shown in Table 3.

TABLE 3
RT-PCR reaction thermal cycling parameters

| Temperature/time | Function |
|---|---|
| 48° C. for 30 minutes | Reverse Transcription |
| 95° C. for 10 minutes | Taq polymerase activation |
| 95° C. for 15 seconds | Denature |
| 60° C. for 1 minute | Anneal/Extend |

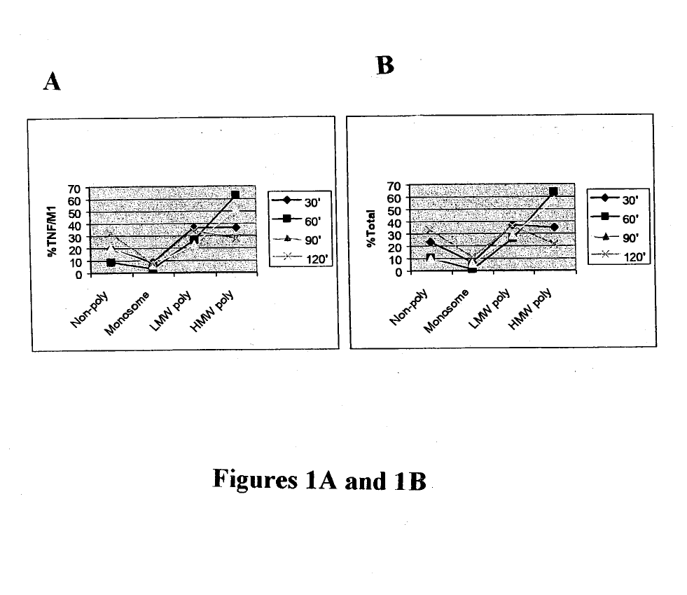

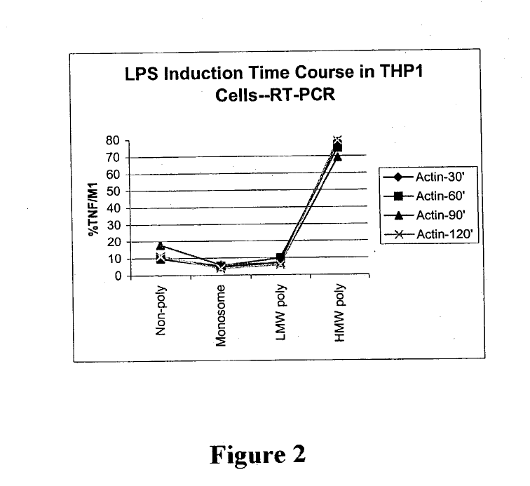

The number of PCR cycles (i.e., the last two steps listed in Table 3) was 35. The reaction mixtures in the 96 well plate were loaded in the Gene Amp 5700 Sequence Detection System (ABI). A standard curve was generated for each run and used to calculate the quantity of the PCR product or amplicon for each reaction. The data are expressed in FIGS. 1A and 2 as a percent of the total RNA found in all the fractions across the sucrose gradient. For comparison to the results obtained in three days using the methods of the present invention, FIG. 1B contains the amount of TNFα RNA measured in three weeks using traditional two-step precipitation and Northern blot analysis of a polysome distribution gradient.

Because the RNA purified by the methods of the present invention can be analyzed by RT-PCR, the amount of heparin and sucrose remaining in these samples must be below the inhibitory amounts calculated in Example 1 (e.g., below 0.004% sucrose and 0.012 ng/μL heparin). This decrease corresponds to the removal of at least 99.7% of the sucrose and at least 99.9% of the heparin from the sucrose gradient fractions. If desired, the purification procedure can be repeated one or more times to further reduce the level of sucrose and/or heparin in the purified RNA solution, further increasing the efficiency of subsequent amplification reactions. As an alternative to the column purification methods described herein or as an additional purification step performed before or after a column purification method, the RNA-containing solution can be inserted in a dialysis membrane with an appropriate pore size to retain the RNA but allow the sucrose and/or heparin to pass through the membrane into the surrounding dialysis buffer. If desired, a control RNA can be also be inserted into the dialysis membrane to account for any possible lose of RNA due to binding to the membrane or passage through the membrane.

If desired, the amount of residual sucrose and/or heparin in the purified RNA solution can be measured using standard methods. For example, radiolabeled sucrose or heparin can be added to the sucrose gradient fractions before purification and the radioactivity of the purified RNA solution can be measured to determine the amount of sucrose or heparin remaining after purification. The amount of sucrose can also be determined enzymaticly, as described previously by Velterop and Vos (Phytochem. Anal. 12:299-304, 2001). Other exemplary methods for measuring the amount of heparin in a sample include those described by Mielke et al. (Clin. Appl. Throm. Hemost. 5:267-276, 1999); Flom-Halvorsen et al. (Ann. Thorac. Surg. 67:1012-1016, 1999); and Hansen et al. (Anesth. Anal. 91:533-538, 2000).

### EXAMPLE 3

Identifying UTRs that Modulate Translation Efficiency

As discussed above, in eukaryotes, translational regulation usually occurs at the initiation step. Cis-elements in the 5′ UTR, as well as the 3′ UTR are important for regulating translation initiation. The cis-elements can be used in assays to identify UTRs that affect the level of translation efficiency of one or more RNA molecules in a cell.

In one such method, a nucleic acid sequence containing a desired RNA regulatory element, for example, a partial or full-length 5′ or 3′ UTR is operatively linked to a reporter nucleic acid. The reporter nucleic acid may include, but is not limited to, nucleic acids encoding luciferase, β-galactosidase, chloramphenicol acetyltransferase, green fluorescent protein, and any RNA sequence that is not normally found in the cell in which the level of RNA translation efficiency is being measured, but for which expression can be measured. The sequence may also include any other desired regulatory elements. In addition, a tag, for example a drug resistance marker, may also be incorporated into the nucleic acid sequence. This tag can then be used for identification of cells in which the RNA UTR/reporter nucleic acid sequence is stably integrated.

The above-described RNA UTR/reporter nucleic acid sequence, generally contained in a vector, is stably or transiently transfected into a cell of interest. Exemplary cells that can be used to measure translation efficiency include cancer cells and any other cells associated with a disease state. If desired, stable transfectants are then selected, for example, by drug selection if a selectable marker is incorporated into the vector. Alternatively, any method known to one skilled in the art may be used to identify and select for stably or transiently transfected cells.

The polysome distribution analysis of RNA isolated from the cells is performed as described herein. The amount of reporter RNA associated with high molecular weight (HMW) polysomes is directly proportional to the level of RNA translation efficiency. If a UTR sequence mediates lower levels of RNA translation efficiency, then the reporter RNA is distributed throughout the sucrose gradient, or the reporter RNA is primarily or only associated with only low molecular weight (LMW) polysomes, which are contained in a different fraction than the HMW polysomes.

### EXAMPLE 4

Identifying Regions in RNA UTRs that Alter Translation Efficiency

Regions in RNA molecules that alter the level of RNA translation efficiency are identified by making reporter constructs, as described herein, consisting of successively smaller fragments of a larger RNA UTR nucleic acid molecule operatively linked to a reporter nucleic acid. Alternatively, reporter constructs are made in which a RNA UTR nucleic acid molecule contains one or more mutations or deletions. These reporter constructs may also contain any other desired regulatory elements. The reporter constructs are transformed into a cell, to generate stably or transiently transfected cells. The effect of the RNA molecules on the level of RNA translation efficiency is then measured, either alone or in the presence of a candidate compound if so desired, using methods described herein. By comparing the translation efficiency of the truncated or mutated RNA nucleic acid molecules to the levels of translation efficiency mediated by full-length or wild-type RNA nucleic acid molecules, the region(s) of RNA nucleic acid molecules involved in mediating translation efficiency can be identified.

### EXAMPLE 5

Screening for Compounds that Modulate RNA Translation Efficiency

Identification of compounds that modulate RNA translation efficiency can be accomplished by administering one or more candidate compounds to cells or an in vitro sample to be tested for translation efficiency. Candidate compounds that modulate the level of translation efficiency can be identified by comparing the translation efficiency of RNA isolated from a cell or sample exposed to the candidate compound with the translation efficiency of RNA isolated from a control cell or sample not exposed to the candidate compound or exposed to vehicle only. Candidate compounds that modulate the translation efficiency are identified if the levels of translation efficiency in the two samples differ. A compound can directly modulate RNA translation efficiency, for example, by binding to the RNA. Alternatively, a candidate compound can indirectly modulate translation efficiency of RNA by interacting with one or more proteins that bind to the RNA and altering an RNA binding protein's ability to participate in the translation of the RNA. In addition, a candidate compound can alter the expression of a protein involved in translation of the RNA, thereby modulating RNA translation efficiency. These methods may be performed to select compounds the modulate the translation of any RNA of interest or the translation of a reporter gene operably linked to one or both UTRs from an RNA of interest. Assays measuring RNA translation efficiency of a control cell can be performed separately from, or together with, the assays of the test cells. When performed separately, the control cell assay can be performed either before, after, or during the test cell assays.

If desired, RNA binding proteins can be added to the assay to determine the effect of a combination of a candidate compound and an RNA binding protein on RNA translation efficiency. RNA binding proteins can be identified and isolated according to the methods described in U.S. Ser. No. 09/165,868, filed Oct. 2, 1998. Desirably, the RNA binding protein is administered to, or expressed in, the cell or sample under conditions that allow interaction between the RNA UTR and the RNA binding protein, and such conditions are known in the art. Identification of a candidate compound that modulates RNA translation efficiency differently when in the presence of an added RNA binding protein may provide information regarding the mechanism of action of the candidate compound. In addition, the interaction between the RNA binding protein and the RNA UTR may occur through direct interaction, or it may be mediated by one or more proteins. The identification of the protein involved in a binding interaction can be identified using standard molecular biology techniques, such as those described in U.S. Ser. No. 09/165,868, filed Oct. 2, 1998.

Compounds identified as modulators of RNA translation efficiency can be used to affect the function or translation of RNA in a cell in vivo or ex vivo. The identification of such compounds can also lead to the development of therapies to treat or prevent a number of disease or conditions, for example, cancer and other proliferative diseases.

### EXAMPLE 6

Therapeutic Applications of Identified Modulators of Translation Efficiency

A compound identified as capable of modulating RNA translation efficiency by any of the above-described methods may be administered within a pharmaceutically acceptable diluent, carrier, or excipient, in unit dosage form. Conventional pharmaceutical practice may be employed to provide suitable formulations or compositions to administer the identified compound to patients suffering from a proliferative disease. Methods well known in the art for making formulations are found, for example, in “Remington: The Science and Practice of Pharmacy” (19th ed., ed. A. R. Gennaro AR., 1995, Mack Publishing Company, Easton, Pa.). If desired, treatment with a compound identified according to the methods described above, may be combined with more traditional therapies for a proliferative disease, for example, traditional chemotherapeutic agents, radiation therapy, or surgery. In addition, these methods may be used to treat any subject, including mammals, humans, domestic pets, or livestock.

The criteria for assessing response to therapeutic modalities employing an identified compound is dictated by the specific condition and will generally follow standard medical practices. Generally, the effectiveness of administration of the compound can be assessed by measuring changes in characteristics of the disease condition. By a “dosage sufficient to modulate translation efficiency” is meant an amount of a compound or that increases or decreases the translation of one or more RNA molecules of interest when administered to a subject. Desirably, for a compound that decreases translation efficiency, protein expression is decreased by at least 10%, 30%, 40%, 50%, 75%, or 90% more in a treated subject than in the same subject prior to the administration of the inhibitor or than in an untreated, control subject. A compound that increases translation efficiency desirably increases the amount of protein expression at least 1.5-, 2-, 3-, 5-, 10-, or 20-fold more in a treated subject than in the same subject prior to the administration of the modulator or than in an untreated, control subject.

Other Embodiments

From the foregoing description, it will be apparent that variations and modifications may be made to the invention described herein to adopt it to various usages and conditions.

All publications and patent applications mentioned in this specification are herein incorporated by reference to the same extent as if each independent publication, patent, or patent application was specifically and individually indicated to be incorporated by reference.

What is claimed is:

1. A method of amplifying an RNA molecule from a polysome distribution sucrose gradient, said method comprising the steps of:
(a) purifying an RNA molecule in a solution from a polysome distribution sucrose gradient under conditions that reduce the amount of heparin in said solution by at least 90%; and
(b) amplifying said purified RNA molecule.

2. The method of claim 1, further comprising the step of (c) quantitating the amount of said amplified RNA molecule.

3. The method of claim 1, further comprising the step of (c) sequencing said amplified RNA molecule.

4. The method of claim 1, wherein the amount of heparin in said solution is decreased by at least 95%.

5. The method of claim 1, further comprising reducing the amount of sucrose in said solution by at least 90% prior to step (b).

6. The method of claim 5, wherein the amount of sucrose in said solution is decreased by at least 95%.

7. The method of claim 1, wherein the amount of heparin and the amount of sucrose in said solution are decreased simultaneously.

8. The method of claim 1, wherein the amount of said RNA indicates the translation efficiency of said RNA.

9. The method of claim 1, wherein said polysome distribution analysis is performed in the presence of a candidate compound, and wherein a change in the polysome distribution of said RNA effected by said candidate compound indicates that said candidate compound modulates the translation efficiency of said RNA.

10. A method of amplifying an RNA molecule that binds a compound of interest, said method comprising the steps of:
(a) contacting one or more RNA molecules in a solution comprising heparin with a compound of interest under conditions that allow at least one RNA molecule to bind said compound of interest;
(b) purifying said RNA molecule that binds said compound of interest under conditions that reduce the amount of heparin in said solution by at least 90%; and
(c) amplifying said purified RNA molecule.

11. The method of claim 10, wherein said compound of interest is an organic molecule having a molecular weight less than 1000 daltons.

12. The method of claim 10, wherein said compound of interest is a protein.

13. The method of claim 10, further comprising the step of (c) quantitating the amount of said amplified RNA molecule.

14. The method of claim 10, further comprising the step of (c) sequencing said amplified RNA molecule.

15. The method of claim 10, wherein the amount of heparin in said solution is decreased by at least 95%.

16. The method of claim 10, wherein said contacting is performed in the presence of a candidate compound, and wherein a change in the amount of said RNA bound to said compound of interest that is effected by said candidate compound indicates that said candidate compound modulates the binding of said RNA to said compound of interest.

17. The method of claim 1, wherein said RNA molecule is a eukaryotic RNA molecule.

18. The method of claim 1, wherein at least 100 different RNA molecule are amplified.

19. The method of claim 10, wherein said RNA molecule is a eukaryotic RNA molecule.

20. The method of claim 10, wherein at least 100 different RNA molecule are amplified.
